(19) United States
(12) Patent Application Publication
(10) Pub. No.: US 2019/0365286 A1
(43) Pub. Date: Dec. 5, 2019

(54) PASSIVE TRACKING OF DYSKINESIA/TREMOR SYMPTOMS

(71) Applicant: Apple Inc., Cupertino, CA (US)

(72) Inventors: William R. Powers, III, San Francisco, CA (US); Maryam Etezadi-Amoli, Santa Clara, CA (US); Adeeti V. Ullal, Mountain View, CA (US); Daniel Trietsch, Cupertino, CA (US); Sara Kianian, Los Altos, CA (US); Hung A. Pham, Oakland, CA (US)

(73) Assignee: Apple Inc., Cupertino, CA (US)

(21) Appl. No.: 15/996,411

(22) Filed: Jun. 1, 2018

(51) Int. Cl. A61B 5/11; A61B 5/00; A61B 5/0205; G16H 50/20

(52) U.S. Cl. CPC A61B 5/1101; A61B 5/02405; A61B 5/681; A61B 5/0004; A61B 5/1114; A61B 5/7278; A61B 5/7221; A61B 5/1121; A61B 5/0205; A61B 5/749; A61B 5/1127; G16H 50/20; A61B 2562/0219; A61B 2562/0223; A61B 2562/0257; A61B 5/7282

(57) ABSTRACT

Embodiments are disclosed for passive tracking of dyskinesia and tremor symptoms using a wearable computer. In an embodiment, a method comprises: obtaining, by one or more motion sensors of a computer attached to a user's limb, motion data; extracting, by one or more processors of the computer, one or more features from the motion data that are potentially indicative of dyskinesia or tremor; determining, by one or more processors of the computer and based on the one or more extracted features, the likelihood of dyskinesia or tremor; generating, by the one or more processors, data indicating the likelihood of dyskinesia or tremor; and outputting, by the one or more processors, the data through an output device of the computer.

### TECHNICAL FIELD

This disclosure relates generally to mobile applications and wearable computers for managing chronic disease and helping patients better engage with clinicians.

### BACKGROUND

Parkinson's disease (PD) is a chronic, progressive neurodegenerative disease that affects the nerve cells in the brain that produce dopamine (a neurotransmitter). There are an estimated 600,000 to 1 million cases of PD in the United States and 60,000 new cases are diagnosed each year. Symptoms of PD include depression, anxiety, sleep disorders, bradykinesia, rigidity, balance gait deficits, speech deficits, tremor and dyskinesia. The most common form of tremor is resting tremor. Resting tremor is an unintentional movement that affects a limb when it is at rest and stops for the duration of a voluntary movement. Dyskinesia is an uncontrollable and involuntary movement that can resemble twitching, fidgeting, swaying or bobbing. Dyskinesia tends to occur when other PD symptoms, such as tremor, rigidity and bradykinesia, are well controlled. Dyskinesia typically occurs as a complication of the long-term use of levodopa, the most commonly used medicine to treat PD. Dyskinesia commonly manifests as chorea, a repeated, irregular, jerky motion that rises and falls with medication and is one of the most disabling forms of dyskinesia.

Patients with PD are typically treated using dopamine (e.g., levodopa) replacement therapy to help reduce some of the symptoms of PD. Over time, the dopamine replacement therapy becomes less effective and begins to induce more side effects, such as dyskinesia. A patient's quality of life is largely dependent on how precisely clinicians titrate and schedule the patient's medications to minimize the patient's symptoms. This is a challenge for clinicians because each patient has a different combination of symptoms that can change and become more severe over time. Also, in any given day the symptoms may fluctuate based on medications, food intake, sleep, stress, exercise, etc.

Today's clinical standard for treating PD includes a combination of a patient interview with opportunistic in-clinic observations. Because a patient may be asymptomatic during their visit to the clinic, the clinician often relies on the patient's self-reported symptom tracking outside the clinic. Some patients, however, may have difficulty remembering their symptoms since their last visit to the clinic.

In addition to the patient's self-reported symptoms, clinicians use a gold standard scale called the Movement Disorders Society—Unified Parkinson's Disease Rating Scale (UPDRS). UPDRS is comprised of a series of tasks performed by the patient in the clinic under the observation of the clinician and thus only provides a snapshot in time of the patient's symptoms. The clinician observes the displacement of a limb (e.g., displacement of the patient's wrist) and assigns a tremor score based on the observed displacement of the limb. Based on the score the tremor is labeled as normal, slight, mild, moderate or severe. Because the clinician's observation is coarse and subjective, the clinician may require multiple visits with the patient and adjustments to their medication schedule before they find one that is effective in managing the patient's symptoms.

### SUMMARY

Embodiments are disclosed for passive tracking of dyskinesia and tremor symptoms using a wearable computer.

In an embodiment, a method comprises: obtaining, by one or more motion sensors of a computer attached to a user's limb, motion data; extracting, by one or more processors of the computer, one or more features from the motion data that are potentially indicative of dyskinesia or tremor; determining, by one or more processors of the computer and based on the one or more extracted features, the likelihood of dyskinesia or tremor; generating, by the one or more processors, data indicating the likelihood of dyskinesia or tremor; and outputting, by the one or more processors, the data through an output device of the computer.

In an embodiment, a system comprises: one or more motion sensors; one or more processors; memory storing instructions that when executed by the one or more processors, cause the one or more processors to perform operations comprising: obtaining, by the one or more motion sensors, motion data; extracting, by the one or more processors, one or more features from the motion data that are potentially indicative of dyskinesia or tremor; determining, by the one or more processors and based on the one or more extracted features, the likelihood of dyskinesia or tremor; and generating, by the one or more processors, data indicating the likelihood of dyskinesia or tremor.

Other embodiments can include an apparatus, computing device and non-transitory, computer-readable storage medium.

One or more of the disclosed embodiments provide one or more of the following advantages: 1) provides all-day symptom/severity tracking information; 2) provides a clinical tool for evaluating patient response to medication; 3) aides the clinician in titrating medication; 4) tracks disease/symptom progression; 5) helps the clinician understand the effect of activity, exercise and sleep on symptoms (contextualization of symptoms); 6) allows patients to better plan activities around symptom patterns; and 7) gives patients and clinicians a more detailed, quantitative, continuous tracking of symptoms than current clinical standards which use questionnaires and UPDRS.

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIG. 1 illustrates a system for passive tracking of dyskinesia/chorea and tremor using a wearable computer, according to an embodiment.

FIG. 2 is a block diagram illustrating data flows for continuous passive tracking of dyskinesia/chorea and tremor using a wearable computer, according to an embodiment.

FIG. 3A is a plot of acceleration readings output by an accelerometer of a wearable computer, according to an embodiment.

FIG. 3B is a plot of displacement based on the acceleration readings of FIG. 3A to illustrate the tracking of changes in tremor severity, according to an embodiment.

FIG. 4 is a block diagram of a data flow for a time-domain displacement estimate, according to an embodiment.

FIG. 5 is a block diagram of a data flow for a frequency-domain displacement estimate, according to an embodiment.

FIG. 6A is a plot of acceleration data generated by an accelerometer of a wearable computer worn by a user while walking, according to an embodiment.

FIG. 6B is a plot of the spectral entropy for the acceleration data shown in FIG. 6A, according to an embodiment.

FIG. 7A is a plot of acceleration data generated by an accelerometer of a wearable computer worn by a user that is experiencing chorea, according to an embodiment.

FIG. 7B is a plot of the spectral entropy for the acceleration data shown in FIG. 7A, according to an embodiment.

FIG. 8A is a plot of spectral entropy for a user who is walking to illustrate the effect of harmonics on spectral entropy, according to an embodiment.

FIG. 8B is a plot of the spectral entropy of FIG. 8A with folded harmonics, according to an embodiment.

FIG. 9A is a plot of spectral entropy for a user who is experiencing chorea to illustrate the effect of harmonics on spectral entropy, according to an embodiment.

FIG. 9B is a plot of spectral entropy of FIG. 9A with folded harmonics, according to an embodiment.

FIG. 10 is a flow diagram of a process for passive tracking of dyskinesia/chorea and tremor using a wearable computer, according to an embodiment.

FIG. 11 is example wearable computer architecture for a wearable computer implementing the features and operations described in reference to FIGS. 1-10.

### DETAILED DESCRIPTION

### Example System

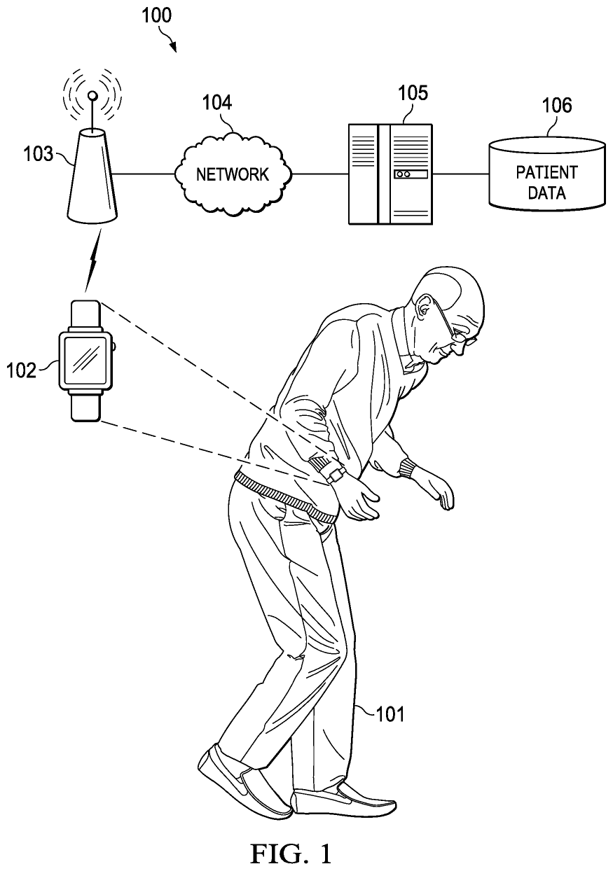

FIG. 1 illustrates a system 100 for passive tracking of dyskinesia/chorea and tremor using a wearable computer, according to an embodiment. System 100 includes wearable computer 102 (e.g., a smart watch) worn on a limb (e.g., a wrist) of user 101 (e.g., a patient with PD). Wearable computer 102 tracks dyskinesia/chorea and tremor and reports tracking data through a display (e.g., LED display) of the wearable computer and/or through a wireless connection (e.g., WiFi, Bluetooth, 5G) to access point 103 (e.g., WiFi router, cell tower, the wearable computer 102 configured as a hot spot). Access point 103 communicates with network 104 (e.g., Internet, WLAN) through a wired or wireless connection. Network 104 communicates with a server computer 105 that is configured to access database 106 of patient data.

In an example embodiment, wearable computer 102 is worn on the wrist of user 101 and passively and continuously tracks symptoms of dyskinesia/chorea and tremor all-day using one or more motion sensors (e.g. tri-axis accelerometer, tri-axis gyroscope) embedded in a housing that can be strapped to a limb of user 101 (e.g., a smartwatch). Output reported on a display of wearable computer 102 (e.g., detection, displacement, frequency) allows user 101 to better understand their disease and medication. For example, if the symptoms are less or more severe during a certain time of day, user 101 can schedule his/her activities around that time of day. The output also allows user 101 to identify the effects of certain activities on their symptoms, such as whether exercise reduces tremor. The output further can help user 101 see the effect of their medications and motivate them to remain adherent to their prescribed regimen.

In an embodiment, output of wearable computer 102 is also reported to medical care providers who can use the output to adjust treatment including but not limited to medication timing, dosage and deep brain stimulation settings. The output can help medical care providers identify or better understand symptoms not observed during appointments at the clinic. In an embodiment, server 105 is operated by a medical clinic and the patient's data is securely stored in database 106. Encrypted data reported by wearable computer can be sent to server 105 using a secure session from any location in the world, thus allowing for continuous passive tracking in between visits to the clinics. A clinician can use the data stored in database 106 to more accurately adjust the medication of user 101 rather than rely solely on their coarse observations and subjective opinion of the patient's symptoms in the clinic. A clinician can also use the data to help them understand how consistent the patient is about taking their medications by observing the consistency of the symptom mitigation compared to what is expected for a given treatment regimen.

Although the disclosure that follows describes a single smartwatch worn on the wrist for tracking symptoms, any medical or consumer device that can be attached to a limb and that includes at least one motion sensor can be used with the disclosed embodiments. Also, the user can wear multiple computers at different locations on their body. For example, the user can wear a computer around their ankle to measure displacement of their ankle. In an embodiment, the motion sensor can be included in an earpiece that is inserted in the user's ear or in glasses (e.g., smart glasses) to measure displacement of the user's head. In an embodiment, the motion sensors (e.g., accelerometers) can provide motion data (e.g., acceleration data) to a physically separate companion device (e.g., a smart phone) for processing and communication to other devices (e.g., to server computer 105). Displacement can be measured by the motion sensors at various locations on the user's body and then combined in the companion device and used to detect symptoms of dyskinesia/chorea and tremor. Displacement may be measured passively (e.g. throughout the day for tremor), or potentially during active tasks (e.g. finger to nose assessments).

In an embodiment, other sensor data from other sensors can be used with the motion data to detect symptoms of dyskinesia and tremor, such as a heart rate monitoring sensor to measure a decrease in heart rate variability (HRV) or an image capture device (e.g., video camera) with a depth sensor (e.g., infrared depth sensor). In the latter, the user takes a video snippet of their symptoms and computer vision algorithms implemented on the wearable computer (or in a server computer) calculate the displacement of a marker (e.g., the smart watch attached to the user's wrist) in the video snippet. The image-based displacement measurement can be used with the motion-based displacement measurement to improve detection of symptoms of dyskinesia/chorea and tremor.

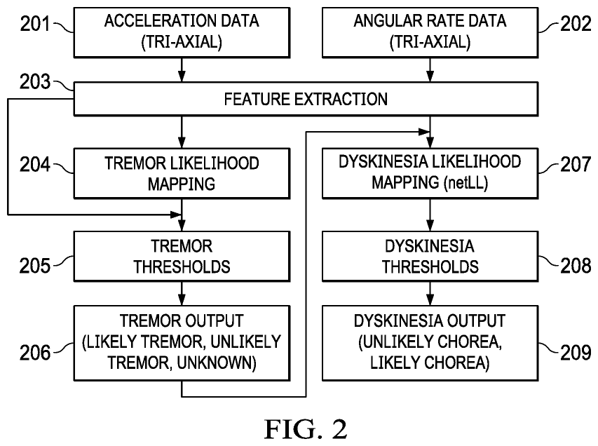

FIG. 2 is a block diagram illustrating data flows for continuous passive tracking of dyskinesia/chorea and tremor using wearable computer 102 of FIG. 1, according to an embodiment. Acceleration data 201 is output by a tri-axial accelerometer of wearable computer 102 at a suitable sample rate (e.g., 100 Hz). Additionally, angular rate data 202 is output by a tri-axial gyroscope of wearable computer 102 at a suitable sample rate (e.g., 100 Hz). Alternate embodiments including filtering the raw accelerometer data 201 or angular rate data 202 to remove noise.

Features are then extracted from these sets of data in process 203. This feature extraction process includes transforming the time-domain data into the frequency domain using an N-point Fast Fourier Transform (FFT). FFT is an algorithm that samples data over a period of time (or space) and divides it into its frequency components. These components are single sinusoidal oscillations at distinct frequencies each with their own amplitude and phase. In this embodiment, a smaller value of N was chosen for tremor features than chorea features because of the high-frequency and intermittent nature of resting tremor. Additionally, the FFT window may be padded or lengthened to improve the frequency resolution.

In process 203, various features are extracted both from the raw time-domain accelerometer and gyro data and from the frequency spectrum generated by the FFTs described above. These features include, but are not limited to: measures of power in different frequency bands, signal-to-noise ratios (SNRs) comparing different frequency bands, frequencies with peak power in a given accelerometer and gyro frequency spectrum (Acc_Max_Frequency, Gyro_Max_Frequency), peak quality, the displacement of the user's wrist (Displacement) and the energy, power or level of movement detected in a frequency range that is less than that of a typical walk cadence (Max_LowFreq_Movement). All or a subset of the foregoing features can be extracted and used to detect dyskinesia, tremor or chorea. The choice of frequency bands can be either fixed (e.g., 3-7 Hz) or tuned depending on prior observations the system has of the individual's symptoms.

In an embodiment, a “support” feature (HasSupport) can be used as a way to gate or improve the reliability or consistency of a tremor call. This “support” feature may require a repeated, consistent max detected frequency or may also be combined with other parameters such as displacement, an individual's past symptom history, an expected phenotype or likelihood based on characteristics of the individual's disease (e.g. years with Parkinson's). Furthermore, this support feature can be but is not limited to a form where it is implemented as a fixed threshold, a likelihood or prior based on a population, or a likelihood or prior designed to dynamically adapt for a given individual. “Support” may also be bolstered by the verification of consistency in frequency across different axes in accelerometer data, or the consistency of frequency across accelerometer or gyroscope data. In an alternate embodiment, additional features include ratios between accelerometer and gyro power.

In process 204, a tremor likelihood is calculated using training data as a function of one or more selected features from 203. This training data can come from an asymptomatic population (e.g., non-PD healthy controls, PD patients without the symptom of interest, age-matched cohort, etc.). This data can be fit with fixed thresholds or an n-dimensional Gaussian/ellipsoid model. In an embodiment, this model can be further extended with other features or sensor inputs (e.g., other accelerometers/gyroscope signals at other sites within the wearable computer even if on the same body location, other inertial sensors at other locations, or other sensors altogether such as proximity sensors). The model thresholds are selected such that that the false positive rate is acceptably low (e.g., 1%). It is possible that via a set duration of observations for a given individual, tremor can be determined to be predominant in either its “rotational” or “translational” components, and thus the SNR, or displacement values for either gyroscope or acceleration features respectively can be weighted differently (or threshold values can also change). These values may also change based on a detected pose. For example, based on whether an individual is determined to be likely seated with arms rested against legs (constrained), in a position with arms free at side, could influence the parameters observed by our sensors. The “pose” or likely hand position can be treated as an input to improve or adjust tremor detection and displacement estimates.

Next, the extracted features resulting from feature extraction 203 and likelihood mapping 204 can be compared to the tremor thresholds 205 to determine a tremor output 206 (e.g., tremor likely, tremor unlikely, or tremor unknown). These thresholds can be fixed thresholds or probabilistic thresholds based on population wide data. Alternatively these thresholds may be individualized thresholds that are set after a period of n days (e.g. n=5-7). Some thresholds like SNR and displacement thresholds only use fixed thresholds to uphold some minimal quality of signal. For example, Acc_Max_Frequency can be tested to determine that it falls within a desired frequency band associated with tremor, the Displacement can be compared against a displacement threshold, SNRs can be compared against fixed SNR thresholds, Max_LowFreq_Movement can be compared to a Max_LowFreq_Movement threshold and HasSupport can be tested to see if it is TRUE. If any of tremor thresholds 205 are false, the algorithm further determines whether tremor is absent (unlikely) or whether a call cannot be made (unknown). This can be done by checking for movement in the low frequency regions to determine whether the subject is at rest and therefore a call can be made reliably.

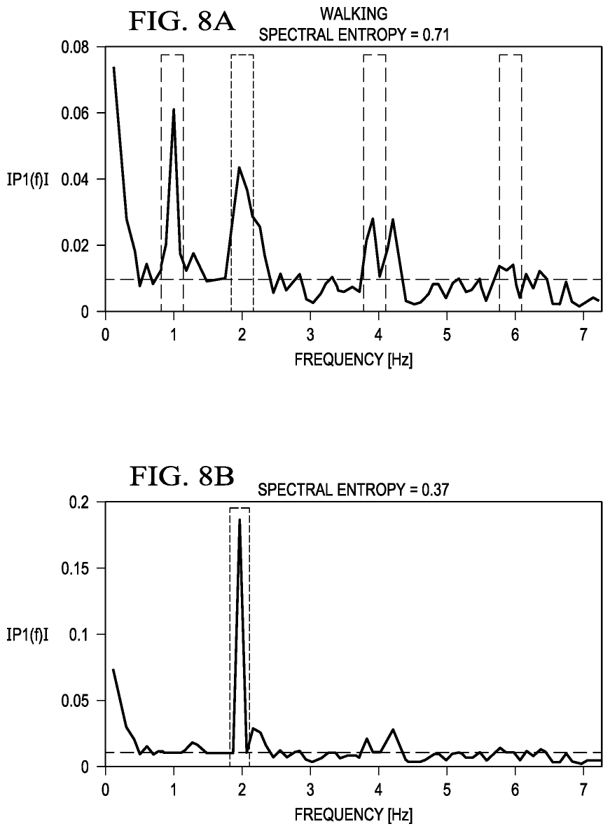

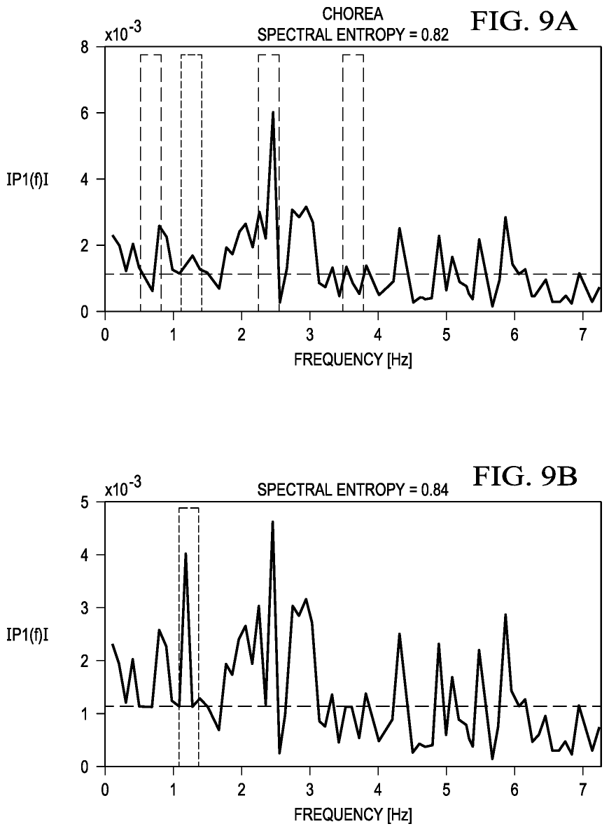

Particular features output by process 203 that are designed to detect dyskinesia/chorea include but are not limited to: the total power in a desired frequency range, harmonics, jerk and spectral entropy. Spectral entropy characterizes the frequency content of the acceleration data and can be used to distinguish dyskinesia/chorea, which is characterized by random and aperiodic movements, from ordinary walking, which is substantially periodic. Harmonics are detected and folded into their “base” frequency (e.g., Acc_Max_Frequency) before calculating spectral entropy, as described in reference to FIGS. 8 and 9. One or more of harmonics detection, jerk detection, power calculations and entropy calculations can also occur at multiple sensor sites. Multiple sensor sites can help improve identification of a walking frequency if one of the sensor locations is anywhere on the lower limb or trunk. In addition multiple sensor locations can capture an undulating (or cascading) movement that is often common in dyskinesia. This undulation may also be captured by gyroscope data.

In an embodiment, spectral entropy can be calculated according to Equations [1]-[3]:

$$\begin{matrix}{P}^{′} ( i ) = F F {T ( i )}^{2} , & [ 1 ] \\ P ( i ) = {P}^{′} / {\sum }_{j} ( {P}^{′} ( j ) ) , & [ 2 ] \\ Entropy = {\sum }_{i} P ( i ) * \frac{{log}_{2} ( P ( i ) )}{{log}_{2} ( k )} , & [ 3 ]\end{matrix}$$

where i varies over k frequencies in the desired range, P′ (i) is the power spectral density and P(i) is the power density function.

In an embodiment, a dyskinesia likelihood mapping (netLL) is calculated 207 by a look-up of the extracted dyskinesia features and the tremor output 206 in a table of probabilities created from training data. This likelihood mapping can be aggregated over a specified time period and compared to the dyskinesia feature thresholds 208 to determine the dyskinesia output 209. If netLL exceeds the threshold value, then chorea is likely. Otherwise, chorea is unlikely. This table of probabilities can be tuned or modified based on one or more of an individual's activity level (e.g., as observed for an initial period of n days), other parameters of their disease (e.g. years with PD, clinician evaluation of UPDRS scores, etc.), typical activities, workouts performed per week or walking speed.

In an embodiment, machine learning can be used to improve tremor and dyskinesia detection based on characteristics of the individual's lifestyle (e.g., user activity level), characteristics of the symptom itself (e.g., frequency, rotational component level) and contextual cues (e.g., position of the user's arm, degree of constraint). In an embodiment, training data for a machine learning algorithm can be divided between people believed to have chorea that affected the wrist their device was worn on and people believed not to have dyskinesia. The table of probabilities that informs dyskinesia can be changed based on an output of the machine learning algorithm (e.g., support vector machines, linear regression, k-means clustering, neural networks, etc.), such as an individual's activity level and/or detected presence of bradykinesia (as indicated by slowness in movement or active tasks). Additionally, tremor detection/estimation can be improved by learning and bounding characteristics of the tremor including by not limited to any combination of: tremor's characteristic frequency; tremor's characteristics displacement; characteristics rotational vs. translational motion; accounting for changes in pose or constrained position in the above characteristics; and accounting for time of day in the above metrics.

In an embodiment, the report can include tremor and chorea results for each minute that the wearable computer is worn during the day. After the foregoing analysis is completed, a report is generated that includes detection results for tremor (likely, unlikely, unknown), displacement (cm) and maximum frequency (Hz). The report also includes a report on detection results for dyskinesia/chorea (likely, unlikely). The report is encrypted and stored in memory of the wearable computer. The report can be displayed on a display of the wearable computer in text and/or graphics. The report can also be transferred through a secure wired or wireless connection to another device or to a network server computer over a secure session, such as a server computer operated by a medical clinic. This allows the clinician to analyze the data and to determine a better treatment plan either through lifestyle changes, dosage changes/titration or deep brain stimulation.

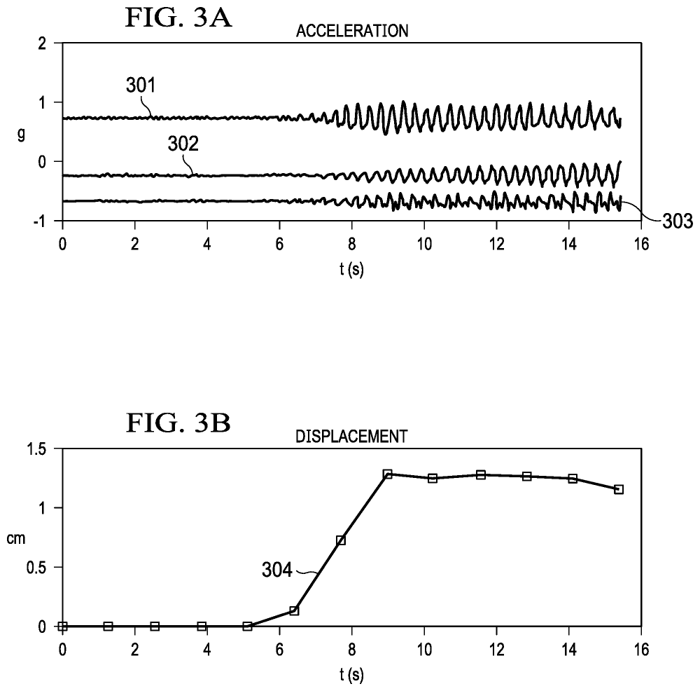

FIG. 3A is a plot of acceleration readings output by a tri-axial accelerometer of a wearable computer, according to an embodiment. Readings 301, 302, 303 correspond to y, x, z-axes, respectively. These readings are generated by a tri-axial accelerometer embedded in a smartwatch worn on the user's wrist. The displacement measured from the acceleration readings is the displacement of the user's wrist in centimeters (cm). Between t=6s and t=15s the patient experiences a tremor symptom. FIG. 3B is a plot of the wrist displacement 304 based on the acceleration readings of FIG. 3A, illustrating how the displacement tracks the change in tremor severity.

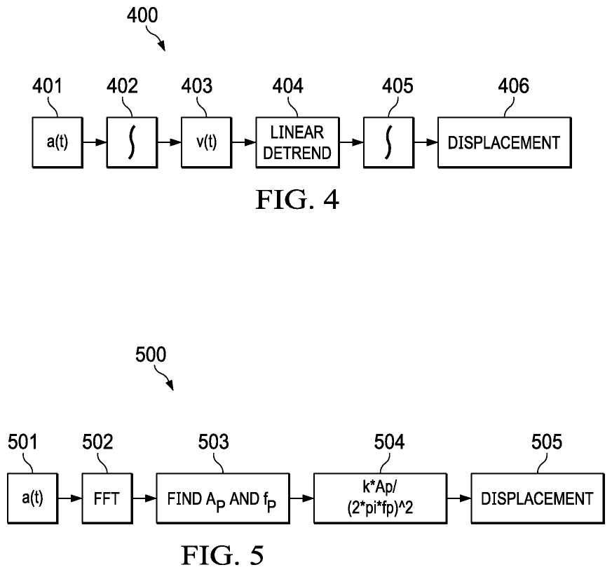

FIG. 4 is a block diagram of data flow 400 for a time-domain displacement estimate, according to an embodiment. Data flow 400 starts with acceleration data 401 in the time domain (a(t)) being integrated 402 to obtain velocity data 403 in the time-domain (v(t)). A linear detrend 404 is applied to velocity data 403. Linear detrend 404 removes the mean value or best straight-line fit from velocity data 403. Next, velocity data 403 is integrated 405 to provide displacement 406 in the time-domain. A problem with data flow 400 is that artifacts can be created in displacement 406 due to detrending failures.

FIG. 5 is a block diagram of data flow 500 for a frequency-domain displacement estimate, according to an embodiment. Acceleration data 501 in the time-domain (a(t)) is transformed into the frequency-domain using FFT 502. The peak power Aₚ and corresponding frequency fₚ in the frequency spectrum is determined 503 from the output of FFT 502. The peak power Aₚ and corresponding frequency fₚ are used in frequency-domain integration 504 by assuming that the amplitude to Aₚ and the frequency to fₚ are the amplitude and frequency of a pure-tone sinusoid waveform. In an embodiment, a closed form equation for the double integral in the Fourier domain is used:

$$displacement \approx \frac{k \cdot {A}_{p}}{{( 2 π \cdot {f}_{p} )}^{2}} , \qquad [ 4 ]$$

where k is a scaling factor to convert to units of displacement (e.g., cm). The output of Equation [4] is an estimate of the displacement amplitude 505, assuming a pure-tone sinusoidal acceleration waveform. The frequency-domain displacement estimate 505 has fewer artifacts then the time-domain displacement estimate 406 resulting from dataflow 400 shown in FIG. 4.

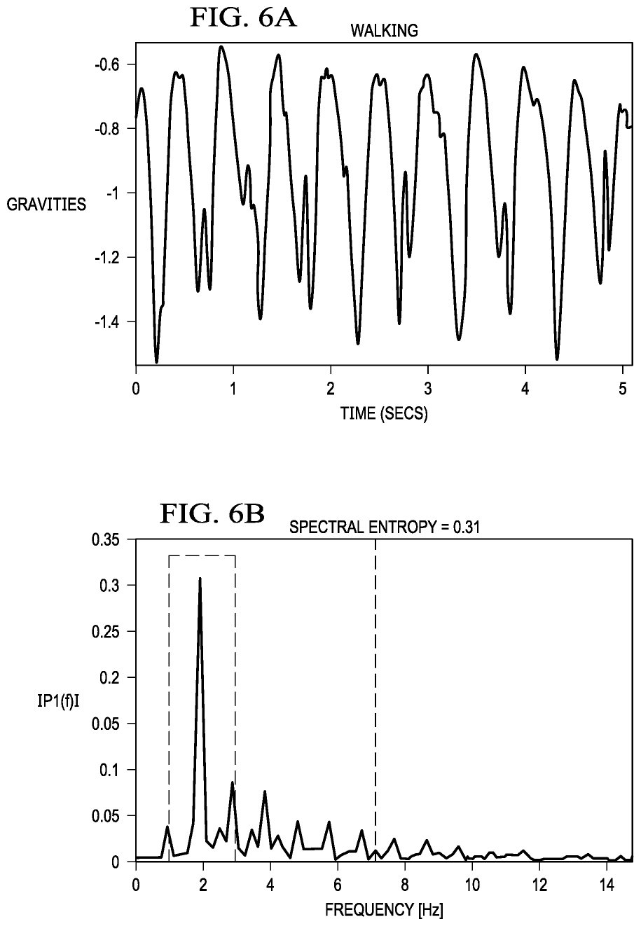

FIG. 6A is a plot of acceleration data generated by an accelerometer of a wearable computer worn by a user while walking, according to an embodiment. FIG. 6B is a plot of the spectral entropy for the acceleration data shown in FIG. 6A, according to an embodiment. As previously stated, spectral entropy can be used to distinguish dyskinesia/chorea, which is characterized by random and aperiodic movements, from ordinary walking, which is substantially periodic. In this example, a desired frequency range of interest (e.g., 0-7 Hz as defined by the vertical dashed line) includes a large peak around 1.8 Hz, which is within the typical walking frequency range. The spectral entropy is 0.31, which is low due to the periodicity of walking.

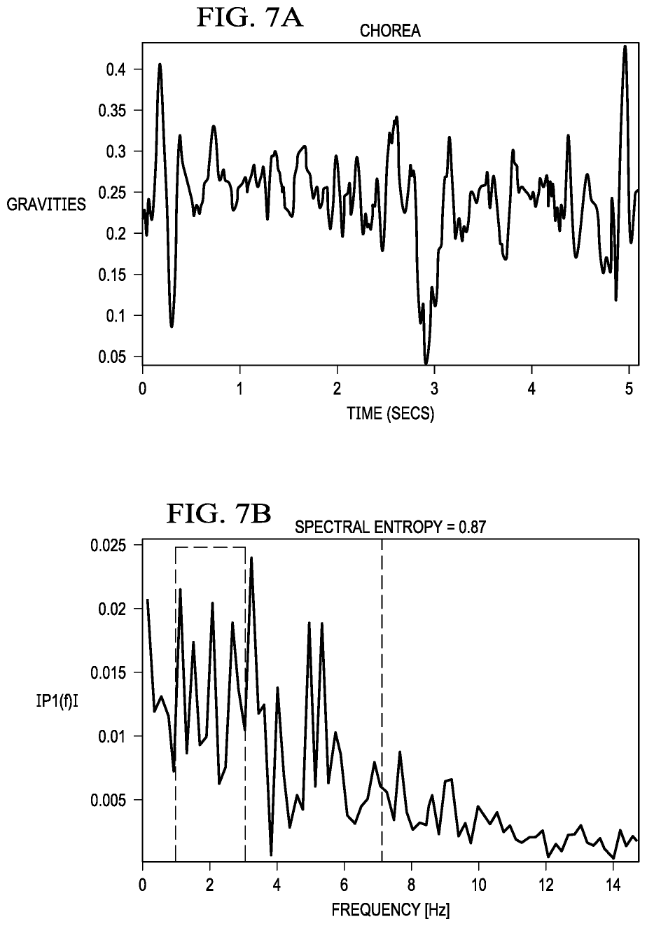

FIG. 7A is a plot of acceleration data generated by an accelerometer of a wearable computer worn by a user that is experiencing chorea, according to an embodiment. FIG. 7B is a plot of the spectral entropy for the acceleration data shown in FIG. 7A, according to an embodiment. If we look at the same frequency range of interest (0-7 Hz) we do not see a single peak that has most of the power due to the aperiodicity of chorea. In this example, the spectral entropy is 0.87, which is much higher than the spectral entropy for walking.

FIG. 8A is a plot of spectral entropy for a user who is walking to illustrate the effect of harmonics on spectral entropy, according to an embodiment. FIG. 8B is a plot of the spectral entropy of FIG. 8A with folded harmonics, according to an embodiment. In FIG. 8A, the calculation of spectral entropy results in multiple power peaks that are fairly close in magnitude due to harmonics. The harmonics result in a spectral entropy of 0.71, which suggests that the acceleration data is aperiodic even though the acceleration data was generated while the user was walking. To address this issue the harmonic energy can be reassigned to the base frequency (e.g., Acc_Max_Frequency) as described in reference to FIG. 2. In an embodiment, a particular harmonic pattern (e.g., 0.5, 1.0. 2.0, 3.0) expressed as multiples of the base frequency is chosen and then the base frequency is selected as the frequency within a selected range (e.g., 1-3 Hz) that maximizes the power captured around the harmonic frequencies. After reassignment of the harmonic energy to the base frequency (˜1.8 Hz), the spectral entropy is 0.37, which is more consistent with walking.

FIG. 9A is a plot of spectral entropy for a user who is experiencing chorea to illustrate the effect of harmonics on spectral entropy, according to an embodiment. FIG. 9B is a plot of spectral entropy of FIG. 9A with folded harmonics, according to an embodiment. In these figures, we can see that even though there are multiple power peaks due to harmonics, reassigning the harmonic energy to the base frequency (˜1.2 Hz) does not affect the spectral entropy for chorea in the frequency range of interest. Folding the harmonic energy to the base frequency results in a spectral entropy of 0.84, which is close to spectral entropy of 0.82 when the harmonic energy is not folded to the base frequency. Accordingly, the reassignment of the harmonic energy to the base frequency allows the periodic motion caused by walking to be differentiated from the aperiodic motion caused by chorea, and thus reduce the number of false positives of detection of chorea.

### Example Process

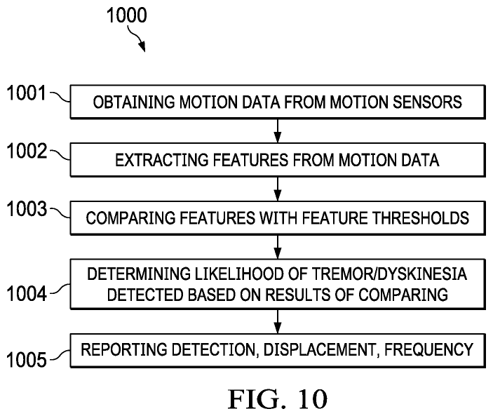

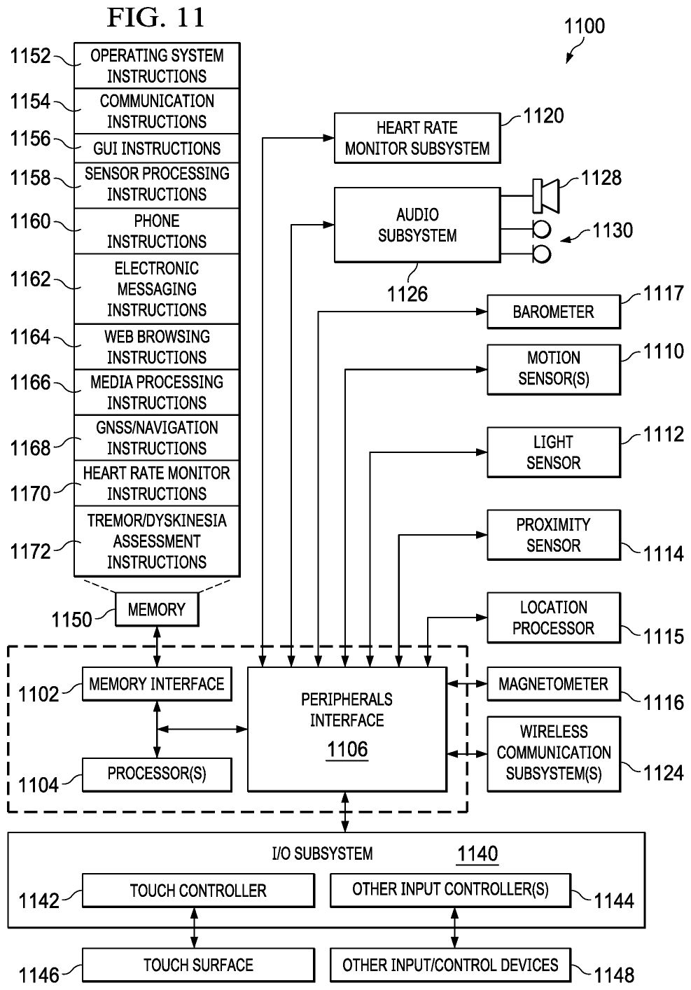

FIG. 10 is a flow diagram of a process 1000 for passive tracking of dyskinesia and tremor using a wearable computer, according to an embodiment. Process 1000 can be implemented by architecture 1100, as described in reference to FIG. 11.

Process 1000 begins by obtaining motion data from motion sensors (1001). For example, acceleration data is obtained from a tri-axial accelerometer and angular rate data is obtained from a tri-axial gyroscope. In an embodiment, the raw acceleration data can be processed to remove gravitational acceleration or filtered (e.g., with a high-pass filter) to remove noise.

Process 1000 continues by extracting features from the motion data (1002). For example, the features can be extracted from the frequency spectrum of the acceleration data and angular rate data. An N-point FFT (e.g., 512, 1024) is used to transform windowed (e.g., rectangular, Hanning or Hamming window functions) samples of the acceleration data and angular rate data into their respective frequency components. Features can then be extracted from the frequency spectrums, as described in reference to FIG. 2.

Process 1000 continues by comparing the extracted features with thresholds (1003), and then determining a likelihood that tremor or dyskinesia is detected based on results of the comparing (1004). In an embodiment, features extracted from the frequency spectrum of the angular rate data are used to determine the likelihood of false positives. In an embodiment, the detection can be confirmed by comparing features over multiple sample. For example, if at least one other sample in +/−N samples (e.g., 4 sample) shares the same Acc_Max_Frequency, then the detection is confirmed.

Process 1000 continues by reporting detection results (likely, unlikely, unknown) for tremor, displacement (cm) and maximum frequency (Hz) for tremor and detection results (likely, unlikely) for dyskinesia/chorea (1005). The report can be presented on a display of the wearable computer or transmitted over a secure wired or wireless network to another device, or to a server computer operated by a medical clinic. In an embodiment, the data are encrypted while stored in memory on the wearable computer. In an embodiment, to further protect privacy the encrypted data are retrieved from the wearable computer by a radio frequency identification (RFID) reader in the medical clinic after the reader is authenticated.

### Exemplary Wearable Computer Architecture

FIG. 11 illustrates example wearable computer architecture 1100 implementing the features and operations described in reference to FIGS. 1-110. Architecture 1100 can include memory interface 1102, one or more data processors, image processors and/or processors 1104 and peripherals interface 1106. Memory interface 1102, one or more processors 1104 and/or peripherals interface 1106 can be separate components or can be integrated in one or more integrated circuits.

Sensors, devices and subsystems can be coupled to peripherals interface 1106 to provide multiple functionalities. For example, one or more motion sensors 1110, light sensor 1112 and proximity sensor 1114 can be coupled to peripherals interface 1106 to facilitate motion sensing (e.g., acceleration, rotation rates), lighting and proximity functions of the wearable computer. Location processor 1115 can be connected to peripherals interface 1106 to provide geopositioning. In some implementations, location processor 1115 can be a GNSS receiver, such as the Global Positioning System (GPS) receiver. Electronic magnetometer 1116 (e.g., an integrated circuit chip) can also be connected to peripherals interface 1106 to provide data that can be used to determine the direction of magnetic North. Electronic magnetometer 1116 can provide data to an electronic compass application. Motion sensor(s) 1110 can include one or more accelerometers and/or gyros configured to determine change of speed and direction of movement of the wearable computer. Barometer 1117 can be configured to measure atmospheric pressure around the mobile device. Heart rate monitoring subsystem 1120 can be configured to measure the heartbeat of a user who is wearing the computer on their wrist. In an embodiment, subsystem 1120 includes LEDs paired with photodiodes for measuring the amount of light reflected from the wrist (not absorbed by the wrist) to detect a heartbeat.

In an embodiment, a digital image capture device and a depth sensor (both not shown) can be coupled to peripherals interface 1106. The digital image capture device (e.g., a video camera) captures images (e.g., digital photos, video clips) and depth sensor (e.g., infrared, LIDAR) capture depth data (e.g., point cloud data) for rendering three-dimensional scenes for augmented reality (AR) and virtual reality (VR) applications. In an embodiment, the digital image capture device can be used for localization using, for example, visual odometry.

Communication functions can be facilitated through wireless communication subsystems 1124, which can include radio frequency (RF) receivers and transmitters (or transceivers) and/or optical (e.g., infrared) receivers and transmitters. The specific design and implementation of the communication subsystem 1124 can depend on the communication network(s) over which a mobile device is intended to operate. For example, architecture 1100 can include communication subsystems 1124 designed to operate over a GSM network, 3G, 4G, 5G, a GPRS network, an EDGE network, a WiFi™ network, near field (NF) and a Bluetooth™ network. In particular, the wireless communication subsystems 1124 can include hosting protocols, such that the mobile device can be configured as a base station for other wireless devices.

Audio subsystem 1126 can be coupled to a speaker 1128 and a microphone 1130 to facilitate voice-enabled functions, such as voice recognition, voice replication, digital recording and telephony functions. Audio subsystem 1126 can be configured to receive voice commands from the user.

I/O subsystem 1140 can include touch surface controller 1142 and/or other input controller(s) 1144. Touch surface controller 1142 can be coupled to a touch surface 1146. Touch surface 1146 and touch surface controller 1142 can, for example, detect touch contact and movement (gestures) or break thereof using any of a plurality of touch sensitivity technologies, including but not limited to capacitive, resistive, infrared and surface acoustic wave technologies, as well as other proximity sensor arrays or other elements for determining one or more points of contact with touch surface 1146. Touch surface 1146 can include, for example, a touch screen or the digital crown of a smart watch. I/O subsystem 1140 can include a haptic engine or device for providing haptic feedback (e.g., vibration) in response to commands from processor 1104. In an embodiment, touch surface 1146 can be a pressure-sensitive surface.

Other input controller(s) 1144 can be coupled to other input/control devices 1148, such as one or more buttons, rocker switches, thumb-wheels, infrared ports, Thunderbolt® ports and USB ports. The one or more buttons (not shown) can include an up/down button for volume control of speaker 1128 and/or microphone 1130. Touch surface 1146 or other controllers 1144 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 1146; and a pressing of the button for a second duration that is longer than the first duration may turn power to the mobile device on or off. The user may be able to customize a functionality of one or more of the buttons. The touch surface 1146 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 1102 can be coupled to memory 1150. Memory 1150 can include high-speed random access memory and/or non-volatile memory, such as one or more magnetic disk storage devices, one or more optical storage devices and/or flash memory (e.g., NAND, NOR). Memory 1150 can store operating system 1152, such as the iOS operating system developed by Apple Inc. of Cupertino, Calif. Operating system 1152 may include instructions for handling basic system services and for performing hardware dependent tasks. In some implementations, operating system 1152 can include a kernel (e.g., UNIX kernel).

Memory 1150 may also store communication instructions 1154 to facilitate communicating with one or more additional devices, one or more computers and/or one or more servers, such as, for example, instructions for implementing a software stack for wired or wireless communications with other devices. Memory 1150 may include graphical user interface instructions 1156 to facilitate graphic user interface processing; sensor processing instructions 1158 to facilitate sensor-related processing and functions; phone instructions 1160 to facilitate phone-related processes and functions; electronic messaging instructions 1162 to facilitate electronic-messaging related processes and functions; web browsing instructions 1164 to facilitate web browsing-related processes and functions; media processing instructions 1166 to facilitate media processing-related processes and functions; GNSS/Location instructions 1168 to facilitate generic GNSS and location-related processes and instructions; and heart rate monitoring instructions 1170 to facilitate heart rate monitoring. Memory 1150 further includes tremor/dyskinesia assessment instructions 1172 for performing the features and processes described in reference to FIGS. 1-10.

Each of the above identified instructions and applications can correspond to a set of instructions for performing one or more functions described above. These instructions can be implemented as separate software programs, procedures, or modules or as a single body of code. Memory 1150 can include additional instructions or fewer instructions. Various functions of the mobile device may be implemented in hardware and/or in software, including in one or more signal processing and/or application specific integrated circuits.

The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., SWIFT, Objective-C, C#, Java), including compiled or interpreted languages, and it can be deployed in any form, including as a stand-alone program or as a module, component, subroutine, a browser-based web application, or other unit suitable for use in a computing environment.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, one aspect of the present technology is the gathering and use of data available from various sources to improve the delivery to users of invitational content or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, home addresses, or any other identifying information.

The present disclosure recognizes that the use of such personal information data, in the present technology, can be used to the benefit of users. For example, the personal information data can be used to deliver targeted content that is of greater interest to the user. Accordingly, use of such personal information data enables calculated control of the delivered content. Further, other uses for personal information data that benefit the user are also contemplated by the present disclosure.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services. In another example, users can select not to provide location information for targeted content delivery services. In yet another example, users can select to not provide precise location information, but permit the transfer of location zone information.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publically available information.

What is claimed is:

1. A method comprising:
obtaining, by one or more motion sensors of a computer attached to a user's limb, motion data;
extracting, by one or more processors of the computer, one or more features from the motion data that are potentially indicative of dyskinesia or tremor;
determining, by one or more processors of the computer and based on the one or more extracted features, the likelihood of dyskinesia or tremor;
generating, by the one or more processors, data indicating the likelihood of dyskinesia or tremor; and
outputting, by the one or more processors, the data through an output device of the computer.

2. The method of claim 1, wherein the computer is embedded in a housing configured to be worn on a wrist of the user and the one or more motion sensors include at least one of an accelerometer or a gyroscope.

3. The method of claim 2, wherein the one or more extracted features includes a displacement of the user's wrist.

4. The method of claim 3, wherein a first motion sensor of the one or more motions sensors is an accelerometer, the motion data includes acceleration data output by the accelerometer, and extracting the one or more features further comprises:
determining a first frequency spectrum of the acceleration data;
determining a first base frequency within a first specified frequency range of the first frequency spectrum, wherein the first specified frequency range is associated with a dyskinesia or tremor frequency range.

5. The method of claim 4, wherein determining the likelihood of dyskinesia or tremor further comprises:
comparing the displacement with a displacement threshold value;
comparing power at the first base frequency to power at one or more other frequencies in the first frequency spectrum; and
determining the likelihood of dyskinesia or tremor based at least in part on results of the comparing.

6. The method of claim 5, wherein determining the likelihood of dyskinesia or tremor further comprises:
determining a false likelihood as a function of the first frequency spectrum and a probability distribution of dyskinesia or tremor data; and
determining that the likelihood of dyskinesia or tremor is unknown based at least in part on the false likelihood.

7. The method of claim 6, wherein a second motion sensor of the one or more motion sensors is a gyroscope, the motion data includes angular rate data output by the gyroscope and extracting the one or more features further comprises:
determining a second frequency spectrum of angular rate data measured about an axis aligned with the user's wrist; and
determining a second base frequency within a second specified frequency range of the second frequency spectrum, wherein the second specified frequency range is associated with the dyskinesia or tremor frequency range.

8. The method of claim 7, further comprises:
determining, by the one or more processors, a likelihood of non-dyskinesia by comparing power at the second base frequency to power at one or more other frequencies in the second frequency spectrum, and determining the likelihood of non-dyskinesia based at least in part on results of the comparing.

9. The method of claim 1, where extracting the one or more features further comprises:
determining, by the one or more processors, a spectral entropy of the motion data; and
determining, by the one or more processors, a likelihood of dyskinesia or chorea based at least in part on a likelihood mapping of the spectral entropy to a probability distribution of dyskinesia data.

10. The method of claim 9, wherein the motion data is acceleration data from an accelerometer and determining the spectral entropy further comprises:
determining a frequency spectrum of the acceleration data;
determining a base frequency within a specified frequency range of the frequency spectrum, wherein the specified frequency range is associated with dyskinesia or chorea;
reassigning energy from harmonics of the base frequency to the base frequency; and
determining the spectral entropy at the base frequency.

11. The method of claim 11, wherein determining the likelihood of dyskinesia or tremor further comprises:
determining, based on a machine learning process, the likelihood of dyskinesia or tremor using training data indicative of at least one of the user's activity level, one or more symptom characteristics or one or more contextual clues.

12. A system comprising:
one or more motion sensors;
one or more processors;
memory storing instructions that when executed by the one or more processors, cause the one or more processors to perform operations comprising:
obtaining, by the one or more motion sensors, motion data;
extracting, by the one or more processors, one or more features from the motion data that are potentially indicative of dyskinesia or tremor;
determining, by the one or more processors and based on the one or more extracted features, the likelihood of dyskinesia or tremor; and
generating, by the one or more processors, data indicating the likelihood of dyskinesia.

13. The system of claim 12, wherein the system is embedded in a housing configured to be worn on a wrist of the user and the one or more motion sensors include at least one of an accelerometer or a gyroscope.

14. The system of claim 13, wherein the one or more extracted features includes a displacement of the user's wrist.

15. The system of claim 14, wherein a first motion sensor of the one or more motions sensors is an accelerometer, the motion data includes acceleration data output by the accelerometer, and extracting the one or more features further comprises:
determining a first frequency spectrum of the acceleration data;
determining a first base frequency within a first specified frequency range of the first frequency spectrum, wherein the first specified frequency range is associated with a dyskinesia or tremor frequency range.

16. The system of claim 15, wherein determining the likelihood of dyskinesia or tremor further comprises:
comparing the displacement with a displacement threshold value;
comparing power at the first base frequency to power at one or more other frequencies in the first frequency spectrum; and
determining the likelihood of dyskinesia or tremor based at least in part on results of the comparing.

17. The system of claim 16, wherein determining the likelihood of dyskinesia or tremor further comprises:
determining a false likelihood as a function of the first frequency spectrum and a probability distribution of dyskinesia or tremor data; and
determining that the likelihood of dyskinesia or tremor is unknown based at least in part on the false likelihood.

18. The system of claim 17, wherein a second motion sensor of the one or more motion sensors is a gyroscope, the motion data includes angular rate data output by the gyroscope and extracting the one or more features further comprises:
determining a second frequency spectrum of angular rate data measured about an axis aligned with the user's wrist; and
determining a second base frequency within a second specified frequency range of the second frequency spectrum, wherein the second specified frequency range is associated with the dyskinesia or tremor frequency range.

19. The system of claim 18, further comprises:
determining, by the one or more processors, a likelihood of non-dyskinesia by comparing power at the second base frequency to power at one or more other frequencies in the second frequency spectrum, and determining the likelihood of non-dyskinesia based at least in part on results of the comparing.

20. The system of claim 18, where extracting the one or more features further comprises:
determining, by the one or more processors, a spectral entropy of the motion data; and
determining, by the one or more processors, a likelihood of dyskinesia or chorea based at least in part on a likelihood mapping of the spectral entropy to a probability distribution of dyskinesia data.

21. The system of claim 20, wherein the motion data is acceleration data from an accelerometer and determining the spectral entropy further comprises:
determining a frequency spectrum of the acceleration data;
determining a base frequency within a specified frequency range of the frequency spectrum, wherein the specified frequency range is associated with dyskinesia or chorea;
reassigning energy from harmonics of the base frequency to the base frequency; and
determining the spectral entropy at the base frequency.

22. The system of claim 12, wherein determining the likelihood of dyskinesia or tremor further comprises:
determining, based on a machine learning process, the likelihood of dyskinesia or tremor using training data indicative of at least one of the user's activity level, one or more symptom characteristics or one or more contextual clues.
